(19) United States
(12) Patent Application Publication
(10) Pub. No.: US 2009/0087486 A1
(43) Pub. Date: Apr. 2, 2009

(54) Foam Wafer Containing a Polyvinyl Alcohol-Polyethyleneglycol-Graft Copolymer

(75) Inventor: Markus Krumme, Randolph, NJ (US)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andernach (DE)

(21) Appl. No.: 12/086,283

(22) PCT Filed: Dec. 4, 2006
(86) PCT No.: PCT/EP2006/011610

§ 371 Date: Jun. 9, 2008

(30) Foreign Application Priority Data
Dec. 8, 2005 (DE) 10 2005 058 569.8

(51) Int. Cl. A61K 9/20

(52) U.S. Cl. 424/465; 427/2.14

(57) ABSTRACT

Sheet-like dosage forms dissolving or disintegrating in an aqueous medium for releasing at least one active substance in a body orifice or body cavity. The sheet-like dosage forms comprise a polymer matrix in the form of a solidified foam containing spaces or cavities, as well as at least one pharmaceutical or cosmetic active substance. The polymer of the polymer matrix is a polyvinyl alcohol-polyethylene glycol graft copolymer. Methods for producing such dosage forms are also provided.

### CROSS-REFERENCE TO RELATED APPLICATIONS

This application is a National Stage application of International Application No. PCT/EP2006/011610, filed on Dec. 4, 2006, which claims priority of German application number 10 2005 058 569.8, filed on Dec. 8, 2005, both of which are incorporated herein by reference in their entireties.

### BACKGROUND OF THE INVENTION

1. Field of the Invention

The present invention relates to dosage forms for administering active substances. More particularly, the present invention relates to dosage forms for dissolving or disintegrating in an aqueous medium for releasing at least one active substance, and methods for producing such dosage forms.

2. Description of the Prior Art

For administering active substances via the oral mucosa, buccal or sublingual tablets which release the active substance in the oral cavity are normally utilised. Compared to other peroral dosage forms, absorption of the active substance via the oral mucosa has the advantages, for example, that even patients having difficulty swallowing can be administered medicaments orally, that the onset of action is quick because the intestinal passage is avoided, and that the utilisation of the active substance is high.

As an alternative dosage form to the known buccal and sublingual tablets, sheet-like, wafer-like dosage forms are known which are called “wafers.”

U.S. Pat. No. 5,529,782 describes a rapidly dissolvable device of soluble polymer material or complex polysaccharides, mainly for administration of contraceptives. This device is to have a thickness of 3 to 4.5 mm and the solubility thereof is to be adjustable such that it will have dissolved within 5 to 60 seconds following its administration. It is intended that this device should also be provided in the form of a laminate comprising cavities that are formed by foaming with gas.

EP 0 450 141 B2 is a carrier material for administering medicaments and which rapidly dissolves upon contact with saliva. This carrier material is a porous, dehydrated, skeleton-like carrier substance. More particularly, the carrier material is a skeleton-like carrier substance based on proteins and polysaccharides. The cavities created by dehydration are used for introducing liquid active substances. The gelatine-polysaccharide carriers described in the above prior printed publication can also be used in the form of wafers. No measures are indicated for reducing the tendency of such wafers to adhere, although there is a danger of such adherence occurring since the dehydrated carrier substances are rehydrated at the latest upon coming into contact with saliva, and their surface is thereby rendered adhesive.

WO 98/26764 describes an active substance-containing and film-shaped dosage form which dissolves rapidly upon contact with a liquid and wherein a fat-soluble phase is distributed in the form of liquid droplets in an outer water-soluble phase.

WO 00/18365 describes an edible film which is intended to be rapidly dissolving but which is also able to adhere well to the oral mucosa in order to deliver antimicrobial substances and to reduce the number of unwanted microorganisms in the oral flora. These antimicrobial substances are ethereal oils which are mixed as the lipophile phase with the aqueous phase, which preferably contains pullulan as the matrix material.

US 2001/006677 discloses film-like, effervescent and water-soluble or water-swellable dosage forms that readily adhere to the oral mucosa.

WO 02/02085 describes rapidly disintegrating dosage forms for releasing active substances in the oral cavity or in other body orifices. The dosage forms comprise a matrix which contains at least one water-soluble polymer as the base substance and is provided with cavities.

WO 2004/060298 describes rapidly dissolving films for oral administration of pharmaceutical active substances, comprising a polyvinyl alcohol-polyethylene glycol graft copolymer and an active substance.

WO 2005/009386 discloses rapidly dissolving films which can be used for oral application of cosmetic or pharmaceutical active substances. These films are based on a polyvinyl alcohol-polyethylene glycol graft copolymer.

Because of their sheet-like form and smooth surface, the known wafers have a tendency to adhere and stick firmly to the palate or to other surfaces of the mucous membrane in the oral cavity, even if they have not been designed as mucoadhesive dosage forms. This disadvantageous effect occurs particularly with comparatively thick wafers as the disintegration time of a wafer is, inter alia, dependent on its thickness, and thicker wafers disintegrate more slowly than thin ones. As a consequence, especially in the case of comparatively thick wafers, the perception of the sticky pulpy film forming from the superficially dissolving polymer layers is particularly important.

When a wafer adheres and sticks firmly to the oral mucosa, the person concerned has an unpleasant or disturbing sensation in the oral cavity which is called “mouthfeel.” To improve the sensation caused by the wafer, it has been proposed in WO 02/02085 to provide a sheet-like dosage form that quickly disintegrates or quickly dissolves in an aqueous medium with spaces or cavities within a polymeric matrix of said dosage form, the contents of said spaces/cavities differing from that of the matrix in terms of its state of aggregation.

It was, however, shown by tests that the “mouthfeel” of a sheet-like dosage form according to WO 02/02085, too, needs improving so as to ensure that even sensitive persons have an unpleasant or disturbing sensation in the oral cavity when taking such a dosage form.

### SUMMARY OF THE PRESENT INVENTION

The task underlying the present invention was therefore to provide a sheet-like dosage form in the form of a solidified foam that rapidly disintegrates or rapidly dissolves in an aqueous medium in order to quickly release at least one pharmaceutical or cosmetic active substance in a body orifice or body cavity, preferably in the oral cavity, without an unpleasant or disturbing sensation being perceived in the oral cavity upon taking said dosage form.

Another disadvantage of the dosage forms which are referred to as wafers or known as solidified foams consists in the time- and energy-consuming process for their manufacture. Thus, in the known methods of manufacture, partially saponified polyvinyl alcohol is usually dissolved in water at temperatures from 80 to 90° C. This process step takes about 2 to 3 hours. In addition, this involves prolonged cooling-off times for the solution, or the requirement of active cooling of the solution before it can be foamed.

Hence, another task underlying the present invention was to provide a method for manufacturing sheet-like dosage forms for releasing active substances in body orifices, which dosage forms are to be present in the form of solidified foams and rapidly disintegrate or rapidly dissolve in an aqueous medium, the method obviating, or at least reducing, the disadvantages of the known methods of production in terms of energy costs and/or process times.

The above tasks are, surprisingly, solved by providing a sheet-like dosage form wherein the polymeric matrix is present in the form of a solidified foam of polyvinyl alcohol-polyethylene glycol graft copolymer, and by providing a method wherein a polyvinyl alcohol-polyethylene glycol graft copolymer is used to produce a solidified foam for said sheet-like administration form containing at least one active substance.

### DETAILED DESCRIPTION OF THE PRESENT INVENTION

The dosage form according to the present invention is a sheet-like dosage form disintegrating or dissolving in an aqueous medium, for releasing at least one active substance in a body orifice or body cavity, the sheet-like dosage form comprising a matrix present in the form of a solidified foam having spaces or cavities, as well as at least one pharmaceutical or cosmetic active substance. In the dosage form according to the invention, the spaces or cavities of the foam are filled with a gas, a gas mixture, a liquid or a liquid mixture. The dosage form according to the invention is characterized in that the polymer of the matrix is a polyvinyl alcohol-polyethylene glycol graft copolymer.

A preferred polyvinyl alcohol-polyethylene glycol graft copolymer is the polyvinyl alcohol-polyethylene glycol graft copolymer sold under the trade name KOLLICOAT® IR (BASF AG, Ludwigshafen, Germany), which consists of 75% polyvinyl alcohol units and 25% polyethylene glycol units.

KOLLICOAT® IR is a water-soluble polymer that can be used as a coating for tablets or as a film former in sprays and transdermal therapeutic systems.

The spaces or cavities of the dosage form according to the invention may be present in the polymer matrix each isolated from the other, preferably in the form of solidified bubbles.

According to another embodiment, the spaces or cavities are connected with one another, preferably by forming a contiguous channel system penetrating the matrix.

The proportion of the spaces or cavities is 5 to 98%, preferably 50 to 80%, relative to the overall volume of the dosage form.

The spaces or cavities are preferably filled with a gas or a gas mixture, more preferably with air. It may, however, be advantageous for the spaces or cavities to contain other gases or gas mixtures. The spaces/cavities are preferably filled with an inert gas, i.e. with a gas or gas mixture that does not react with the other components of the dosage form. Gases which are especially preferred are nitrogen, carbon dioxide and helium, as well as a mixture of these gases or of two of these gases.

According to another embodiment, it is provided that the spaces or cavities are filled with a liquid or a liquid mixture (for example an oil), the liquids not being miscible with the matrix material and not dissolving the polymer skeleton of the matrix. The liquid or liquid mixture may, furthermore, contain one or more pharmaceutical and/or cosmetic active substances.

Because the dosage form according to the present invention is present in the form of dried foam, the intended adherence-reducing effect is achieved without excessively restricting the active substance-absorption capacity of the dosage form.

Another important parameter influencing the properties of the dosage form according to the invention is the diameter of the cavities or bubbles. The bubbles or cavities are preferably produced with the aid of a foaming machine. In this way, the diameter of the bubbles can be adjusted, almost arbitrarily, within a broad range. Thus, the diameter of the bubbles or cavities may be within a range of 0.01 to 50 μm; bubbles/cavities having a diameter of between 0.1 and 10 μm are preferred.

In the simplest embodiment of the invention, the cavities of the dosage form according to the invention are free of active substance. It may, however, be advantageous for the spaces or cavities to contain active substances, auxiliary substances and/or additives in order to be able to achieve certain effects. Especially preferred substances which may be contained in the spaces/cavities are tensides or gas-forming substances by means of which it is possible to accelerate the disintegration of the dosage form after its application.

In addition, as a measure to further reduce the tendency of the dosage forms to adhere to a mucous membrane, the surfaces of the dosage form may be uneven or irregular, preferably wavelike or relief-like, or be provided with a structured surface. An irregular surface structure can be caused, for example, by the bubble-shaped cavities themselves which have been introduced in the polymer matrix, and/or by a subsequent, special drying treatment.

The dosage forms according to the present invention are designed so as to be thin, for example in the form of wafers. The thickness of the dosage form is preferably 0.1 to 5 mm, more preferably 0.5 to 1 mm. The lower limit for the thickness of the dosage forms is about 50 μm.

Suitable as active substances are—without restriction—therapeutically active compounds. These may originate from the following groups: agents for treatment of infections; virostatics; analgesics such as fentanyl, sufentanil, buprenorphine; anaesthetics; anorectics; active substances for treating arthritis and asthma, such as terbutaline; anticonvulsives; antidepressives; antidiabetics; antihistaminics; antidiarrhoeals; agents against migraine, itching, nausea and retching, motion sickness or seasickness, such as scopolamine and ondansetron; anti-Parkinson agents; antipsychotics; antipyretics; spasmolytics; anticholinergics; agents against ulcer, such as ranitidine; sympathomimetics; calcium channel blockers such as nifedipine; beta-blockers; beta-agonists such as dobutamine; antiarrhythmics; antihypertonics such as atenolol; ACE inhibitors such as enalapril; benzodiazepine agonists such as flumazenil; coronary, peripheral and cerebral vasodilators; stimulants for the central nervous system; hormones; hypnotics; immunosuppressants; muscle relaxants; parasympatholytics; parasympathomimetics; prostaglandins; proteins; peptides; psychostimulants; sedatives; tranquilisers.

For administration in the mouth, or to the oral mucous membrane, basically all active substances are suitable which can be absorbed buccally and/or gastrointestinally.

An especially preferred active substance is nicotine. Nicotine can be used here not only in the form of its free base, but also in the form of one or more of its pharmaceutically acceptable salts. Pharmaceutically acceptable salts of nicotine are, for example, nicotine bitartrate, nicotine hydrochloride, nicotine dihydrochloride, nicotine sulfate, nicotine zinc chloride double salt and nicotine salicylate. Likewise, nicotine polacrilin is a potential source of nicotine.

The active substance content per dosage unit is up to 50 mg, preferably up to 30 mg, more preferably up to 20 mg.

Further substances which are suitable as active substances and/or as auxiliary substances are: polishing agents, grinding agents (abrasive), such as titanium dioxide, silicon dioxide, etc.; sodium fluoride, dicalcium phosphate; essential oils such as anise oil, fennel seed oil, eucalyptus oil, peppermint oil, spearmint oil, orange oil, salvia oil, thyme oil, lemon oil, etc.; flavouring agents such as camphor, cineol, eucalyptol, menthol, pinene, cinnamic aldehyde, cinnamic acid, etc.; honey, citric acid, vitamins, antioxidants, sorbite.

The dosage forms according to the present invention are thus also suitable for cosmetic application purposes, as well as for applications in the field of dental care, dental cleaning, oral hygiene or dental hygiene.

Furthermore, the following substances may be contained in the dosage form as flavouring agents, either alone or in combination: vanilla flavour, orange flavour, orange-cream flavour, strawberry flavour, raspberry flavour or chocolate flavour. In addition, one or more sweetening agents may be added, e.g. sucralose, aspartame, cyclamate, saccharin and acesulfame, as well as the salts thereof.

Suitable auxiliary agents are substances from the following group, apart from others well known to those skilled in the art: carboxymethyl cellulose, gum arabic, methyl cellulose, pectins, modified and unmodified starches, gelatine, animal and/or plant proteins, egg albumin, alginates, BRIDGE® or BRIJ® (an emulsifier), isopropanol, benzyl alcohol, ethyl acetate, ethyl citrate, octyl gallate, 1,2-propylene glycate, magnesium stearate, stearic acid, microcrystalline cellulose, aerosil, lecithin, TWEEN®, propyl gallate, amylogam.

Furthermore, a sugar (or a mixture of sugars) or at least one other carbohydrate material may be dissolved in the foam. The sugar or carbohydrate increases the post-drying mass of the foam. In addition, the drying and crystallisation of the sugar or of the other carbohydrate gives the dried foam additional strength and stability. Sugar or other carbohydrates may lead to the sensation of the dried foam having a sweet taste, or they may otherwise improve the organoleptic properties of the foam. Examples for sugars that may be contained in the dosage form are maltose, lactose, saccharose, dextrose (glucose) and trehalose. Sugar alcohols such as mannite, sorbite, xylite, maltite and the like are also suitable. Examples of other suitable carbohydrates are maltodextrins, starch sugar syrup (from maize), soluble starches and the like.

Although the dosage form according to the invention is intended to be used, in particular, for oral application, it is not limited to the administration of active substances in the region of the oral cavity. Rather, the present invention also encompasses dosage forms that are introduced in other body cavities or body orifices, where they are to release the active substances contained therein. Examples to be mentioned in this connection are rectal, vaginal or intranasal dosage forms.

The active substance released from the dosage form is either absorbed at the site of application, e.g. via the oral mucous membrane, or it is transported farther and absorbed at another site (e.g. in the gastrointestinal tract, after swallowing the active substance released in the oral cavity).

The dosage form according to the invention is a preparation which quickly disintegrates or dissolves in aqueous media. The retention time of the inventive dosage form at the application site (e.g. oral cavity), or its disintegration time, is preferably in the range of from 1 second to 5 min., more preferably in the range from 5 seconds to 1 min., and most preferably in the range of 10 seconds to 30 seconds.

Furthermore, during the manufacture of the dosage forms according to the invention, one or more acids may be added in order to give the foam a pleasant sour taste. Examples of such acids include citric acid, lactic acid, acetic acid, benzoic acid, propionic acid, oxalic acid, malonic acid, succinic acid, maleic acid and tartaric acid. The addition of an acid or of acids may furthermore be necessary or desirable in order to lower the pH value of the foam. This is particularly desirable in those cases where the active substance contained in the dosage form is relatively insoluble under alkaline conditions, for example ibuprofen, or where the active substance is not stable under alkaline conditions.

Furthermore, wetting agents or moisturizers may be added to the dosage forms according to the present invention to improve the aesthetic properties of the dried foam and to reduce the fragility or brittleness of the dried foam. Examples of such agents are glycerine, propylene glycol and polyglycerine ester. In addition, it is possible to add surface-active agents prior to or after the drying in order to improve the pre-drying or post-drying stability of the foam. Examples of suitable surface-active agents are, in particular, substituted sorbitan derivatives, preferably those of the “TWEEN®” series (ICI).

Methods of Production

The known methods for producing sheet-like dosage forms in the form of solidified foams are time- and energy-consuming since the process temperature for dissolving partially saponified polyvinyl alcohol in water prior to foaming is usually 80-90° C. and because at this temperature the partially saponified polyvinyl alcohol has to be dissolved by stirring for 2 to 3 hours. Prior to foaming the resultant solution, the latter must be cooled down by observing prolonged cooling-off times or by active cooling.

It was therefore another object of the present invention to provide a method for the production of sheet-like dosage forms which rapidly disintegrate or dissolve in aqueous media, which method obviates the aforementioned disadvantages.

By using a polyvinyl alcohol-polyethylene glycol graft copolymer, it is made possible to carry out the production of the dosage form according to the invention at room temperature, except for the drying of the foamed solution. Dissolving the polyvinyl alcohol-polyethylene glycol graft copolymer in water at room temperature is advantageous in terms of the energy costs and process times as compared to the known methods where the polymer or polymer mixture is dissolved at higher temperatures. In addition, the method according to the invention is also advantageous in terms of the stability of the active substance, especially where the active substance is added to the solvent prior to the polymer.

The following methods are proposed for producing the inventive dosage forms having an improved “mouthfeel”:

In a particularly preferred method of production, first, a solution or dispersion is prepared which contains the polyvinyl alcohol-polyethylene glycol graft copolymer as well as at least one active substance. This solution, which may also be a concentrated solution or a viscous mass, is subsequently foamed by introducing a gas or a gas mixture (e.g. air). This may be done by a dispersing apparatus or a foaming machine, but also by other methods, e.g. by ultrasound. Suitable gases are, in particular, inert gases such as nitrogen, carbon dioxide or helium, or mixtures of inert gases.

To stabilise the foams or the air bubble-containing (or gas bubble-containing) masses thus produced, a foam-stabilising agent can be added before or during foaming. Agents suitable for that purpose, for example tensides, are known to those skilled in the art. Finally, the air bubble-containing mass or the foam is spread as a film or layer on an appropriate support and is subsequently dried. Because the solvent is withdrawn, the foam solidifies during drying and forms an aerogel, during which process the cavities formed receive a permanent structure.

Wafers having the desired surface dimensions or geometric shapes are obtained by casting the foamed coating mass into corresponding moulds, or by punching or cutting the individual wafers out of a piece having a larger surface area.

The active substance-containing dosage forms thus obtained exhibit the properties and advantages of the present invention, which means that they quickly disintegrate after oral application, without causing an unpleasant sensation on the oral mucous membrane.

The shape, number and size of the spaces or cavities created can be influenced by different process parameters, e.g. by the concentration of the polyvinyl alcohol-polyethylene glycol graft copolymer, by the viscosity of the polymer mass, by controlling the foaming process or by the selection of the foam-stabilising agents.

To produce a particularly preferred dosage form which is intended for administration of nicotine, one has to make sure that the nicotine is not present in the foamed solution as a base but as a salt, so that the nicotine does not evaporate during the subsequent drying of the foam. To this end, nicotine may be introduced into the polymer solution in the form of one of its pharmaceutically acceptable salts, for example as nicotine tartrate. As an alternative, the nicotine base may be weighed into the polymer solution and, subsequently, a fruit acid—preferably a fruit acid that is suitable for foods—, which may also serve as a taste masking agent, may be added in a molar excess of 1.4:1, relative to nicotine. Thus, the corresponding nicotine salt is formed and nicotine is prevented from evaporating when the foam dries. Nicotine base would evaporate at the drying temperature of 80° C., which is not the case with the salt.

All fruit acids are suitable for forming the nicotine salt, but citric acid or a dicarboxylic acid, especially maleic acid, succinic acid, fumaric acid and tartaric acid, is used with preference. Mixtures of suitable fruit acids may, however, be used as well.

Another method according to the invention for producing the dosage forms according to the invention provides—as a modification of the above-described method—for the spaces or cavities within the polymer matrix to be formed by introducing a hydrophobic solvent which is immiscible with the solvent used for preparing the above-mentioned solution or dispersion.

An emulsion is formed thereby which contains the hydrophobic solvent in the form of finely distributed droplets. By withdrawing the solvents during the subsequent drying, cavities having the shape of droplets or bubbles remain in the polymer matrix. With a two-phase system, the solvent must first be withdrawn from the internal phase.

Furthermore—as an alternative to the first of the above-described methods—said cavities may be formed in such a way that auxiliary substances are added to the polymer-containing and active substance-containing solution which form a gas or gases, thereby foaming the mass. This foaming by gas formation may either take place during production of the polymer mass or during the coating of the mass onto the support, or as late as during the subsequent drying process. Substances or substance mixtures suitable for gas formation are known to those skilled in the art.

Furthermore, foaming may also be brought about by expanding a previously dissolved gas.

The gas used is preferably an inert gas such as nitrogen, carbon dioxide or helium, or a mixture thereof.

Alternatively, to produce the dosage forms according to the present invention one may start with a melt of the matrix polymer or of the polymer mixture. The processing is in principle similar to that of hot melt coating compounds known from the prior art.

A gas or gas mixture is introduced into the above-mentioned polymer melt by using one of the afore-mentioned methods in order to cause foaming of the melt. Subsequently, the melt is spread onto an appropriate support or extruded or cast into a mould, and then left to cool, i.e. to solidify. Processing from the melt is out of the question if the active substance used is unstable or volatile at the melting temperature of the polymer melt. If necessary, auxiliary substances may be added to the polymer melt to reduce its melting point.

According to a further modification of the above-described methods of production, the polymer matrix is first produced in the form of a block. Subsequently, i.e. after drying or solidification has taken place, the desired sheet-like dosage forms are severed from the block by cutting.

The dosage forms according to the present invention are advantageously suitable for the administration of medicaments in the oral cavity or for rectal, vaginal or intranasal administration. They can be used in human medicine as well as in veterinary medicine.

### EXAMPLE 1

Summary of a Dosage Form According to the Present Invention

| Ingredients | Content (percent by weight) |
|---|---|
| KOLLICOAT ® IR | 67.50 |
| Nicotine bitartrate | 17.90 |
| Peppermint flavour | 11.75 |
| Menthol | 2.55 |
| Sucralose | 0.285 |
| Colourant Blue #1 | 0.015 |

### EXAMPLE 2

Production of a Dosage Form According the Present Invention

To produce a dosage form according to the invention, KOLLICOAT® IR was dissolved in water (30 min, with stirring, at room temperature), and the remaining additives were added. Using a foaming machine, air was introduced into the composition, which composition was subsequently applied to a support and dried at 80° C.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A sheet-like dosage form which dissolves or disintegrates in an aqueous medium, for releasing at least one active substance in a body orifice or body cavity, said sheet-like dosage form comprising a polymer matrix in the form of a solidified foam containing spaces or cavities, as well as at least one pharmaceutical or cosmetic active substance, wherein the polymer of said polymer matrix is a polyvinyl alcohol-polyethylene glycol graft copolymer.

2. The sheet-like dosage form according to claim 1, wherein said polyvinyl alcohol-polyethylene glycol graft copolymer comprises 75% polyvinyl alcohol units and 25% polyethylene glycol units.

3. The sheet-like dosage form according to claim 1, wherein the spaces or cavities in the matrix are isolated from one another and are present in the form of bubbles.

4. The sheet-like dosage form according to claim 1, wherein the spaces or cavities in the polymer matrix are connected with one another for forming a channel system penetrating the polymer matrix.

5. The sheet-like dosage form according to claim 1, wherein said spaces or cavities are filled with air or with a gas.

6. The sheet-like dosage form according to claim 1, wherein said spaces or cavities are filled with a liquid or a liquid mixture, and wherein said liquid or liquid mixture is immiscible with the matrix material.

7. The sheet-like dosage form according to claim 6, wherein said liquid or liquid mixture contains at least one active substance.

8. The sheet-like dosage form according to claim 1, wherein the volume percentage of said spaces or cavities is 5 to 98% relative to the overall volume of the dosage form.

9. The sheet-like dosage form according to claim 1, wherein the surface of the dosage form is unevenly or irregularly shaped.

10. The sheet-like dosage form according to claim 1, wherein wherein said sheet-like dosage form is designed as a wafer, and wherein the thickness of said sheet-like dosage form is between 50 μm and 5 mm.

11. The sheet-like dosage form according to claim 1, wherein at least one of said matrix and said spaces or cavities contains a compound selected from the group consisting of auxiliary substances and additives.

12. A method for producing a sheet-like dosage form according to claim 1, said method comprising the steps of:
a) preparing a solution containing at least one polyvinyl alcohol-polyethylene glycol graft copolymer and at least one active substance;
b) adding a foam-stabilising agent to said solution and foaming the solution by a process selected from the group consisting of introducing a gas or a gas mixture, chemical gas formation, and expanding a dissolved gas, wherein said step of adding a foam-stabilising agent is done before or during said step of foaming the solution, or not at all;
c) spreading the foamed solution onto a coating support to form a coated solution; and
d) drying and withdrawing the solvent to solidify the coated solution.

13. A method for producing a sheet-like dosage form according to claim 1, comprising the steps of:
a) preparing a solution containing at least one polyvinyl alcohol-polyethylene glycol graft copolymer and at least one active substance;
b) adding a hydrophobic solvent which is immiscible with the solvent used for preparing the solution, and preparing an emulsion containing the hydrophobic solvent in the form of finely distributed droplets;
c) spreading the emulsion onto a coating support to form a coated emulsion; and
d) drying and withdrawing the solvent to solidify the coated emulsion.

14. A method for producing a sheet-like dosage form according to claim 1, comprising the steps of:
a) preparing a solution containing at least one polyvinyl alcohol-polyethylene glycol graft copolymer and at least one active substance;
b) adding an auxiliary substance or a combination of auxiliary substances capable of forming a gas;
c) spreading the solution onto a coating support to form a coated solution; and
d) drying and withdrawing the solvent to solidify the coated solution.

15. A method for producing a sheet-like dosage form according to claim 1, comprising the steps of:
a) preparing a polymer-containing melt (hot melt) containing at least one polyvinyl alcohol-polyethylene glycol graft copolymer and at least one active substance;
b) adding a foam-stabilising agent to said melt and foaming the melt by a process selected from the group consisting of introducing a gas or a gas mixture, chemical gas formation, and expanding a dissolved gas, wherein said step of adding a foam-stabilising agent may be done before or during said step of foaming the melt;
c) spreading the melt onto a coating support to form a film; cooling said film; and
d) solidifying the film after said cooling step.

16. The method according to claim 12, further comprising the following steps, wherein said steps c) and d) are replaced or modified by the following steps e) and f):
e) preparing the polymer matrix in the form of a block, starting from said solution, emulsion or dispersion, or from said melt; and
f) cutting the solidified block in order to obtain sheet-like shapes.

17. A method of using a dosage form according to claim 1, comprising the step of administering pharmaceutical active substances or cosmetic active substances in the oral cavity.

18. A method of using the dosage form according to claim 1, comprising the step of administering pharmaceutical active substances to humans or animals in a manner selected from the group consisting of rectal, vaginal and intranasal.

19. The sheet-like dosage form according to claim 5, wherein said gas is an inert gas.

20. The sheet-like dosage form according to claim 19, wherein said inert gas is selected from the group consisting of nitrogen, carbon dioxide, helium, a mixture of nitrogen, carbon dioxide and helium and a mixture of two of nitrogen, carbon dioxide and helium.

21. The sheet-like dosage form according to claim 8, wherein the volume percentage of said spaces or cavities is 50 to 80% relative to the overall volume of the dosage form.

22. The sheet-like dosage form according to claim 9, wherein the surface of the dosage form comprises a shape selected from the group consisting of a wave-like shape and a relief-like shape.

23. The sheet-like dosage form according to claim 10, wherein the thickness of said sheet-like dosage form is between 0.5 and 1 mm.

24. The sheet-like dosage form according to claim 11, wherein said auxiliary substances and additives are at least one selected from the group consisting of tensides and gas-forming substances.

25. The method according to claim 13, further comprising the following steps, wherein said steps c) and d) are replaced or modified by the following steps e) and f):
e) preparing the polymer matrix in the form of a block, starting from said solution, emulsion or dispersion, or from said melt; and
f) cutting the solidified block in order to obtain sheet-like shapes.

26. The method according to claim 14, further comprising the following steps, wherein said steps c) and d) are replaced or modified by the following steps e) and f):
e) preparing the polymer matrix in the form of a block, starting from said solution, emulsion or dispersion, or from said melt; and
f) cutting the solidified block in order to obtain sheet-like shapes.

27. The method according to claim 15, further comprising the following steps, wherein said steps c) and d) are replaced or modified by the following steps e) and f):
e) preparing the polymer matrix in the form of a block, starting from said solution, emulsion or dispersion, or from said melt; and
t) cutting the solidified block in order to obtain sheet-like shapes.

28.-35. (canceled)
